(19) United States
(12) Patent Application Publication
(10) Pub. No.: US 2015/0250889 A1
(43) Pub. Date: Sep. 10, 2015

(54) HEAT-SENSITIVE AMPHIPHILIC POLYURETHANE AND AQUEOUS SOLUTION CAPABLE OF BEING INJECTED BASED ON SUCH MATERIAL

(71) Applicant: GELTIS S.r.l., Torino (IT)

(72) Inventors: Gianluca Ciardelli, Pisa (IT); Susanna Sartori, Alessandria (IT); Monica Boffito, Capriata D'Orba (AL) (IT); Piero Serafini, Torino (IT)

(21) Appl. No.: 14/417,736

(22) PCT Filed: Jul. 11, 2013
(86) PCT No.: PCT/IT2013/000196

§ 371 Date: Jan. 27, 2015

(30) Foreign Application Priority Data
Jul. 27, 2012 (IT) TO2012A000669

(51) Int. Cl. A61K 47/34; A61K 9/00; A61L 27/26; A61L 27/18; A61L 27/52

(52) U.S. Cl. CPC A61K 47/34; A61L 27/18; A61L 27/52; A61L 27/26; A61K 9/0019

(57) ABSTRACT

A heat-sensitive sol-gel composition capable of being injected is described, composed of an aqueous solution of at least one amphiphilic polyurethane.

The present invention refers to heat-sensitive aqueous solutions capable of being injected, based on amphiphilic polyurethane, in particular to be used in the bio-medical field.

Within regenerative and aesthetic medicine, systems capable of being injected are the ideal solution to perform minimally invasive interventions and for obtaining systems for the local and/or controlled release of drugs. Minimally invasive surgical techniques allow reducing problems connected to traditional interventions, with high hospital costs (long-lasting interventions, prolonged patient stay) and long patient healing times (long rehabilitation periods, high risk of post-surgery complications). The local release of drugs allows minimising the side effects due to drugs themselves.

A necessary requirement for systems capable of being injected is being in low-viscosity liquid or gel form, upon injection. Such materials are particularly interesting for regenerative and aesthetic medicine applications, and for the controlled release of drugs, if they form highly viscous gels under physiologic conditions. Similar systems have been obtained mostly through in vivo reticulation or polymerisation procedures. These procedures however have limits connected to the possible use of scarcely bio-compatible monomers or reticulating agents. Moreover, some of these reactions are eso-thermal and generate a local temperature increase.

Moreover, currently marketed systems with local and controlled release have been obtained through injection of polymeric solutions, using organic solvents (Atrigel®). These solvents however are incompatible with the use of some classes of molecules (for example of a proteic nature) which can be denaturated under such conditions.

Sol-gel systems of the present invention are a promising alternative in the field of regenerating damages tissues (regenerative medicine) and in the controlled release of drugs, since gelling does not occur through reactive processes, but following the variation of surrounding conditions. Such systems are therefore highly bio-compatible and suitable for encapsulating numerous classes of molecules. Moreover, hydro-gels capable of being injected are promising substrates for tissue engineering, since they have a water content which is comparable with the one of natural tissues, guarantee an efficient mass transfer, are adapted to be easily handled and can be homogeneously additioned with growth factors, cells or drugs. The chemical-physical nature of hydro-gels further guarantees an easy and homogeneous system colonisation by cells and the complete filling of tissue defects, independently from their shape and sizes. Injectability and gelling in situ, moreover, make the gel formation occur in direct contact with the cells: in this way, a deposit is formed, result of the creation of a network, which can compenetrate cells and proteins of the extra-cellular matrix of the treated tissue. A gel is a compound composed of at least two components: one of them (generally a natural or synthetic polymer or a mixture thereof) forms a three-dimensional reticule immersed into a medium composed of the other component (liquid). Depending on the gelling mechanism upon which they incur, hydro-gels capable of being injected that find application in tissue engineering can be classified into physical or reversible gels and chemical or irreversible gels. In particular, in physical or reversible gels, the passage from the solution (sol) state to the gel state occurs by forming weak interactions between the polymeric chains based on hydrophobic interactions, hydrogen bonds, Van der Waals forces or ionic interactions. Physical hydro-gel show ex-vivo a reversible sol-gel transition; a lower transition, which implies the passage from the sol state to the gel state, and an upper transition next to which the gel collapses or shrinks, expelling part or the whole previously absorbed solvent. These transitions can be induced by changes of temperature, ion concentration, solvent composition or of pH. Hydro-gels showing this behaviour are reactive to stimuli, since a change of their state can be induced through variations of conditions of the surrounding environment. Hydro-gels reacting to stimuli are defined as smart, since the sol-gel transition is induced by the physiologic conditions. Such gels, therefore, are spontaneously formed under certain conditions, without requiring the insertion of reticulating agents, which, typically, are toxic and can limit its degradability. As stated, the transition can be induced by different stimuli; depending on the type of stimulus inducing the sol-gel transition, hydro-gels can be distinguished in: (i) heat-sensitive hydro-gels, (ii) hydro-gels sensitive to pH, (iii) hydro-gels sensitive to particular analytes, (iv) hydro-gels based on peptides, and (v) hydro-gels based on amphiphilic polymers.

As known, an amphiphilic (or amphipatic) polymer is composed of alternate hydrophobic and hydrophylic groups. These molecular characteristics make, in an aqueous solvent, the polymeric chains spontaneously aggregate, giving rise to micelles which expose the hydrophilic groups to the outside, namely towards the aqueous medium, and hydrophobic groups inwards, maximising the interactions between hydrophilic domains and external environment, and maximising interactions between hydrophobic groups and water.

As known, every polymeric solution can be characterised by a critical concentration (CGC), starting from which it is possible to observe a transition from the solution state to the gel state and vice versa. The CGC, usually, is inversely proportional to the molecular weight of the used polymer. Some systems show a separation between solvent and gel over a certain temperature; in this cases, it is a syneresys. As regards heat-sensitive hydro-gels, after having established a certain concentration (greater than the CGC), the polymeric solution can be characterised by two temperatures:
LCGT (Lower Critical Gelation Temperature): temperature next to which the sol-gel transition occurs. For bio-medical applications, this temperature must preferably be around the body one. Below the LCGT, there are polymeric solutions, above the LCGT, the gel is formed;
UCGT (Upper Critical Gelation Temperature): temperature at which the inverse gel-sol transition occurs.

After having set a certain concentration, at temperatures lower than LCGT, small-sized micelles are formed, which freely flow in the aqueous medium. Under these conditions, the hydrogen links between hydrophilic segments of the polymeric chain and water are predominant. When increasing the temperature, interactions between hydrophobic segments increase, hydrogen bonds become weaker, and micelle sizes increase due to the hydrophobic bonds being created between the polymeric chains. Provided that hydro-gel concentration is greater than CGC, the application of a further temperature increase, and having exceeded the LCGT, induce the sol-gel transition: micelle sizes grow, the degree of packaging and aggregation among the micelles increases, till a gel is formed. A further temperature increase above the UCGT implies the destruction of micellar structures and the return to the solution state. Modulation of properties of the sol-gel system, in order to obtain the phase transition under adequate physiologic conditions and physical properties, can be performed by acting both on the polymer composition, and on the solution composition. It is, for example, possible to act on the hydrophoby of the polymeric material using for example macrometers with different molecular weight, solution concentration, molecular weight of the polymer, the presence of additives additioned to the formulation (salts, such as, for example, NaCl), solvent choice.

Among polymers used in making hydro-gels for bio-medical applications, those of natural origin (proteins, polysaccharides) have been widely studied, but however do show some problems, among which the risk of transmitting diseases and a quick degrading.

Biodegradable and non-biodegradable polyurethane have also been examined. Examples of such polyurethane are disclosed in U.S. Pat. No. 4,822,827, U.S. Pat. No. 5,254,662, U.S. Pat. No. 5,900,246, US20060051394. In the bio-medical field, biodegradable polyurethane are a valid alternative to natural polymers due to their excellent mechanical properties, good biocompatibility and processability.

Polyurethane are synthesized using, as reagents, a macrodiol, a diisocyanate and possibly a chain extender. The choice of reagents makes characteristics and properties of polyurethane capable of being modulated depending on specifications of a given application.

Soft segments are composed of polyols, generally with molecular weights included between 400 and 5000 Da. Hard segments, instead, are composed of diisocyanates and possibly chain extenders. These latter ones are usually diols or diamines with low molecular weight. An accurate selection of the chain extender allows providing the polyurethane with suitable characteristics of biodegrading, biomimetism (insertion of aminoacid sequences sensitive to enzymatic degrade, like the Ala-Ala sequence, or adhesion sequences, such as, for example, peptide Arg-Gly-Asp), or inserting functional groups to be used in a second step for functionalising the material (N-BOC serinol).

Polyurethane, moreover, can be in vivo subjects with hydrolytic, enzymatic or oxidative degrade, according to the type of monomers used in their synthesis. Degradable polyurethane can be produced by inserting bonds capable of being hydrolised inside the main polymeric chain. The most common method used for inserting bonds capable of being hydrolised inside the polymeric chain is the one providing for the use, as soft segments, of polyols containing blocks capable of being hydrolised, like polylactides and poly(ε-caprolactone). Alternatively, it is possible to insert groups capable of being hydrolised inside the polymer through the hard segment, in particular through the use of chain extenders capable of being hydrolised.

The synthesis of polyurethane for bio-medical applications occurs through a process with one or two stages; this latter one provides for a first step wherein the synthesis of the prepolymer occurs, and during which an excess of diisocyanate is reacted with the polyol. The prepolymer typically has a low molecular weight and the appearance of a very viscous liquid or a to low-melting solid. The following reaction of the prepolymer with the chain extender is the second step of the synthesis and allows obtaining the final polymer with a multi-block structure of the (AB)n type.

No heat-sensitive polymeric sol-gel compositions based on polyurethane are known, nor their combinations with natural polymers.

Therefore, object of the present invention is solving the above prior art problems, by providing an heat-sensitive sol-gel composition capable of being injected based on polyurethane, in particular to be used in the bio-medical field, which does not imply polymerisation reactions or reticulations during or after implants, and therefore does not need the addition in vivo of reticulating agents or monomers, which are potentially sensibilising or toxic.

Another object of the present invention is providing an heat-sensitive sol-gel composition capable of being injected, based on polyurethane, in particular to be used in the bio-medical field, which does not generate local temperature increases.

Moreover, an object of the present invention is providing an heat-sensitive sol-gel composition capable of being injected, based on polyurethane, in particular to be used in the bio-medical field, which is capable of being injected with minimally invasive injection systems.

Another object of the present invention is providing a polyurethane composition, in particular to be used in the bio-medical field, which can be easily packaged as sterile powders to allow its following solubilisation for use, thereby making this operation quick and free from operation complications.

Moreover, an object of the present invention is providing a heat-sensitive sol-gel composition capable of being injected, in particular to be used in the bio-medical field, which shows, together with the verified biocompatibility of polyurethane, the capability of gelling the solutions at temperatures near the physiological one; such gel provides a mechanical support to tissues and organs and at the same time enables their regeneration.

Another object of the present invention is providing a heat-sensitive sol-gel composition capable of being injected, based on polyurethane, in particular to be used in the bio-medical field, which can have degrade times comparable with the tissue regeneration and be made functional with bio-active molecules.

Another object of the present invention is providing a polyurethane sol-gel composition additioned with one or more drugs, able to release the drug in vivo in a local and/or controlled way.

The above and other objects and advantages of the invention, as will result from the following description, are obtained with a heat-sensitive sol-gel composition capable of being injected, in particular to be used in the bio-medical field, as claimed in claim 1. Preferred embodiments and non-trivial variations of the present invention are the subject matter of the dependent claims.

It is intended that all enclosed claims are an integral part of the present description.

It will be immediately obvious that numerous variations and modifications (for example related to shape, sizes, arrangements and parts with equivalent functionality) can be made to what is described, without departing from the scope of the invention, as appears from the enclosed claims.

The present invention will be described in more detail by some preferred embodiments thereof, provided as a non-limiting example.

The present invention therefore refers to a heat-sensitive sol-gel composition capable of being injected composed of an aqueous solution of at least one amphiphilic polyurethane, in particular to be used in the bio-medical field (namely a polymeric composition which is preferably in solution at ambient temperature and which gelifies, by micellar aggregation under physiologic conditions) synthesized by using as monomers/macromers poly-ethers and aliphatic diisocyanates. It must be noted how the choice of materials composing the composition according to the present invention will always be aimed to having post-implant non-toxic materials and/or in vivo degrade.

Polyethylene glycol (PEG) is often used as hydrophilic block in making co-amphiphilic polymers. PEG is a poly-ether characterised by a complete capability of being mixed in water within a wide range of temperatures and molecular weights. It is a material, which has numerous qualities, such as hydrophilicity and biocompatibility, which make it ideal for biomedical applications. Polyurethane and polyurethane-urea based on polyethylene glycol has amphiphilic properties, which make them a valid choice for developing sol-gel systems, which, by combining biodegradability and injectability characteristics, lend themselves to a minimally invasive insertion and are subjected to a gelling process under physiologic conditions.

As will be described below in more detail, the composition according to the present invention belongs to the category of physical or reversible gels and is subjected to a temperature-dependent gelling process. The temperature increase determined by the passage from the external environment to the physiologic one, therefore, generates the transition from the sol state to the gel state, without needing any other stimulus of a chemical or environmental nature.

In particular, polyurethane used in the heat-sensitive sol-gel composition capable of being injected according to the present invention, are synthesized using as reagents at least:
a first amount Q₁ of at least one and maximum two macrodiols containing at least one block composed of polyethylene glycol (PEG), as oligomer or polymer. Preferably, the block of polyethylene glycol (PEG) has a molecular weight Mₙ included between 200 and 5000 Da.
a second amount Q₂ of at least one diisocyanate of the formula OCN—R—NCO, where R is an aliphatic or aliphatic-alicyclic group containing 4 to 26 carbon atoms.

Preferably, diisocyanate is chosen among 1,6-esamethylene diisocyanate, 1,4-butandiisocyanate, 1,4-cicloesamethylene diisocyanate or L-lysine-diisocyanate.

Preferably, the polymerisation reaction occurs in an anhydrous environment (typically in an atmosphere of inert gases, like nitrogen, N₂, or argon Ar).

Preferably, such reaction mixture can comprise at least one solvent such as, for example, 1,2-dichloromethane, tetrahydrofuran, N,N-dimethyl-formamide, 1,2-dichloroethane. It is possible to provide for the use of many solvents, to enable the solubilization of reagents and/or oligomers which are formed during the reaction. Reagents and solvents used in the synthesis should preferably be anhydrous or anhydrided before the polymerisation reaction; the water content is reduced with a suitable method, in order to obtain a percentage of such molecule which is lower than 1% in weight with respect to the reaction mixture. Examples of anhydridification methods are reflow on molecular sieves and distillation.

Preferably, such reaction mixture comprises at least one catalyst, for example, tertiary amines (such as diaminocyclooctane) or organo-metallic compounds (such as dibutyl-tin-dilaurate).

In addition, the reaction mixture for the synthesis of polyurethane used in the heat-sensitive sol-gel composition capable of being injected according to the present invention can comprise at least one third amount Q₃ of at least one chain extender containing two hydroxyl or aminic groups.

Diols or diamines that can be used as chain extenders, for the synthesis of the polyurethane composition according to the present invention, can have various natures; for example, the chain extender can be chosen among:
diols or diamines containing aminoacid sequences, such as for example peptide adhesion sequences (for example Arg-Gly-Asp), shear sequences (for example Ala-Ala) or cell-penetrating peptides;
diols or diamines composed of aminoacid derivatives (such as, for example, ethylic ester of lysine);
diols or diamines containing a protected functional group (such as, for example, N-BOC serinol);
cyclic diols or diamines (such as, for example, cyclohesane-dimethanol).

Preferably:
the percentage in weight of macrodiol in the reaction mixture is included between 20% and 90% in weight (wt);
percentage in weight of diisocyanate in the reaction mixture is included between 80% and 10% in weight (wt);
percentage in weight of the chain extender in the reaction mixture ranges between 0% and 65% in weight (wt).

The first amount Q₁ of macrodiol, the second amount Q₂ of diisocyanate and the third amount Q₃ of chain extender are present in the reaction mixture according to the present invention, in un molar ratio Q₁:Q₂:Q₃ which can preferably range between 1:2:1 and 3:8:5.

The reaction of the hydroxyl groups (—OH) of the macrodiol with the isocyanate groups (—NCO) of the diisocyanate implies the formation of the urethane group (a suitable ratio between equivalents of the two reagents is necessary for obtaining a isocyanate-terminated reaction product). The polymerisation reaction is possibly performed in solvents, preferably in previously listed organic solvents.

Advantageously, the synthesized polyurethane according to the present invention, as described above, can be used for preparing aqueous solutions: in particular, the polyurethane composition according to the present invention can be solubilized in:
water;
saline solution (such as, for example, phosphate buffer, PBS);
glucosyde solution (glucose or dextrose solution);
gluco-saline solution.

The amount Q of polyurethane present in the polyurethane solution of the present invention is preferably included between 1% and 99% weight//volume (more preferably between 1% and 50%).

In addition, the polyurethane solution of the present invention can comprise, in addition to the polyurethane component Q₄, at least one fifth amount Q of a natural polymer, such as carbohydrates and/or proteins (for example, ialuronic acid, gelatine, collagen). The fifth amount Q₅ is included between 0% and 99% (more preferably between 1% and 20%) weight/volume, according to the present invention.

In addition, the polyurethane solution according to the present invention can comprise at least one sixth amount Q₆ of at least one drug or a bioactive molecule, which can therefore be encapsulated in the micellar system and gradually released in vivo, after having injected the composition in human or animal tissues or organs. Preferably, such sixth amount Q₆ is included between 0% and 30% weight/volume (still more preferably between 0% and 20%).

The following Examples include some typical examples related to preparations stages of the polyurethane solutions of the present invention, as described above.

### STAGE 1

Herein below, as an example, the synthesis is described of a polyurethane according to the present invention, obtained through a two-step synthesis process. Reagents used in this synthesis are:
polyethylene glycol (Mₙ=1500 g/mol);
1,6-esamethylen-diisocyanate (HDI);
ciclohexan-dimethanol (CDM).

The synthesis process occurred through the following steps:
a) anhydrification of solvent and reagents: with the use of a ball.-type system and two soxhlet, solvent (1,2-dichloroetane-DCE-) and reagents (macrodiol and chain extender) are anhydrided in an inert atmosphere of N₂ by reflow on molecular sieves. The operation is performed for 8 hours;
b) pre-polymerisation (first step): the first step of the synthesis occurs at the end of the anhydrification. The pre-polymerisation reaction, performed at 85° C. for 150 minutes, provides for the addition of diisocyanate to the solution of macrodiol in DCE (amount of diisocyanate equal to 22.43% of the amount in weight of macrodiol) and of catalytis amounts of catalyst (dibutyl-tin-dilaurate);
c) addition of the chain extender (second step): the second step of the synthesis occurs at ambient temperature and provides for the addition of the chain extender (amount of extender equal to 9.6% of the amount in weight of macrodiol) to the pre-polymer solution. The extension step lasts for a period of 16 hours, at the end of which the reaction is ended with addition of methanol;
d) precipitation and purification: the polymeric solution is added drop-wise to a non-solvent of the polymer (petroleum ether), the system is decanted and the solution separated from the polymer.

The polymer is solubilised in DCE and again precipitated with the addition of a non-solvent (for example petroleum ether). At the end of this second precipitation, decanting/centrifugation of the polymer is performed. The polymer is then separated from the solvent and placed in a vacuum stove at 40° C. for at least 12 hours.

### STAGE 2a

Herein below, as an example, the preparation of a composition according to the present invention is described, with the following composition:
10% wt polyurethane;
3% wt gelatine (Type B gelatine from cow hide);
solvent: PBS (system volume: 3 ml).

The polyurethane used here is the one whose synthesis is included in stage 1.

The composition is prepared complying with the following protocol:
90 mg of gelatin are hot solubilised in 3 ml di PBS
0.3 g of polyurethane are added to the previous solution and solubilised.

### STAGE 2b

Herein below, as an example, the preparation of a formulation according to the present invention is described, having the following composition:
10% wt polyurethane;
3% wt gelatine (Type B gelatine from cow hide);
solvent: PBS (system volume: 3 ml);
adenosine (concentration: 4 mg/ml).

The polyurethane used here is the one whose synthesis is included in stage 1.

The composition is prepared in compliance with the following protocol:
Polyurethane (0.3 g) is solubilised in acetone (10% w/v); drug (adenosine, 12 mg) and PBS are added to the previous solution, in such amounts as to have a volume ratio PBS:acetone=1:20;
the thereby obtained solution is added drop-wise to the PBS volume;
from the thereby prepared solution acetone is removed;
gelatine (90 mg) is added to the solution.

The polyurethane synthesized according to the present invention, have been characterised through:
Molecular Exclusion Chromatography (SEC) to determine the mean molecular weight in number (Mₙ) and in weight (MO: the thereby synthesized polymers result as having Mₙ included between 10.000 and 25.000 Da;
Differential Scan calorimetric Analysis (DSC) to measure the thermal properties: objective of this technique is locating the thermal transitions and the crystallinity degree of polyurethane. Polyurethane synthesized according to the present invention have a crystallinity in the range 25-30%;
Fourier Transform Infrared Spectroscopy (FTIR) to analyse the chemical structure. In detail, from the analysis of the FTIR spectrum, it has been possible to identify the characteristic peaks of the urethane ground, which confirm that the synthesis successfully occurred. In particular, the peak around 1710 cm⁻¹ is characteristic of the stretching of free C═O groups linked to urethane groups. At about 1100 cm⁻¹ the stretching of the CH₂—O—CH₂ group can be observed, characteristic of aliphatic ethers, such as for example PEG. All other peaks, which can be observed on spectra point out a structure in compliance with the forecasts. No peaks related to diisocyanate groups around 2200 cm⁻¹ have further been detected, pointing out that the conversion of such groups has been quantitative and that, consequently, the polymerisation process occurred completely;
surface wettability through the static Contact Angle, to evaluate the surface properties. Polyurethane synthesized according to the present invention show contact angle values ranging between 40° C. and 60° C. The polyurethane according to the present invention is thereby hydrophilic.

The polyurethane sol-gel compositions according to the present invention have been characterised through:
Injectability Tests through 2.5-ml graduated syringes and a 20 Gauge needle. Injectability tests pointed out that the sol-gel formulation of the present invention can be easily charged inside a syringe and flow without difficulty through a 20 Gauge needle;
Rheological Analysis: rheological tests on sol-gel systems according to the present invention have been carried out through a rotary rheometer, to characterise them from the viscosity point of view (at ambient temperature and under physiologic conditions) and to locate the sol-gel transition starting temperature. Strain sweep tests have been carried out first on all composition being studied, in order to correctly choose the distortion to be imposed in all following analyses. Frequency sweep tests have also been performed, to characterise the behaviour of the sol-gel systems depending on the frequency. In particular, these latter tests pointed out that the compositions according to the present invention have a behaviour of the pseudo-plastic type, namely their complex viscosity η* decreases when the frequency increases, this being also able to be exploited to facilitate injecting the sol-gel compositions by the designed release systems. The temperature ramp tests pointed out that the complex viscosity, as well as the storage and loss modules G′ and G″, increase with the temperature; this behaviour is aligned with the gelling process due to the temperature increase. The viscosity values at ambient temperature (25° C.) resulted variable in the range 0.15 to 2.5 Pa*s; such values make the compositions according to the present invention, easily injectable through the use of commercial devices (such as, for example, insulin-type needles). Kinetic tests at 37° C. allowed to point out that the compositions according to the present invention remain in the gel phase (G′>G″) for the whole time interval taken into account, and that the complex viscosity increases also when the system is kept at constant temperature, demonstrating that the gelling process is not only temperature-dependent, but also time-dependent.

The compositions according to the present invention can find application in numerous fields of the regenerative and aesthetic medicine. The proposed composition, in fact, can serve both as filler, and as drug, growth factor and cells vehicle. Such systems are interesting in regenerating numerous tissues, such as, for example, bone, cartilage, miocardium, in addition to micro- and macro-fillers, for cosmetic (dermal fillers) and aesthetic (prostheses) applications.

1. Heat-sensitive sol-gel composition capable of being injected composed of an aqueous solution of at least one amphiphilic polyurethane, wherein said amphiphilic polyurethane is adapted to be synthesized through a polymerisation reaction mixture comprising at least:
a first amount (Q₁) of at least one and maximum two macrodiols containing at least one block composed of polyethylene glycol (PEG) in a form of an oligomer or a polymer;
a second amount (Q₂) of at least one diisocyanate with formula OCN—R—NCO, where R is an aliphatic group containing 4 to 26 carbon atoms.

2. Composition according to claim 1, wherein said reaction mixture comprises at least one solvent.

3. Composition according to claim 2, wherein said solvent is chosen among 1,2-dichloromethane, tetrahydrofurane, N,N-dimethylformamide, 1,2-dichloroethane.

4. Composition according to claim 1, wherein said reaction mixture comprises at least one catalyst.

5. Composition according to claim 4, wherein said catalyst is chosen among tertiary amines or organ-metallic compounds.

6. Composition according to claim 1, wherein said first amount (Q₁) of macrodiol is included between 20% and 90% in weight (wt) of said reaction mixture and said second amount (Q₂) of diisocyanate is included between 80% and 10% in weight (wt) of said reaction mixture.

7. Composition according to claim 1, wherein when the molecular weight of said block of polyethylene glycol (PEG) has a Mₙ included between 200 and 5000 Da.

8. Composition according to claim 1, wherein said diisocyanate is chosen between 1,6-esamethylen diisocyanate, 1,4-butandiisocyanate or 1,4-cycloesamethylen diisocyanate, L-lysine-diisocyanate.

9. Composition according to claim 1, wherein where it is a physical or reversible gel.

10. Composition according to claim 1, wherein said reaction mixture comprises at least one third amount (Q₃) of at least one chain extender containing due hydroxyl or aminic groups.

11. Composition according to claim 10, wherein said chain extender is chosen among diols or diamines containing amino-acid sequences, diols or diamines composed of amino-acid derivatives, diols or diamines containing a protected functional group, cyclic diols or diamines.

12. Composition according to claim 10, wherein the percentage in weight of said third amount (Q₃) of chain extender in said reaction mixture is included between 0% and 65%.

13. Composition according to claim 10, wherein when said first amount (Q₁) of macrodiol, said second amount (Q₂) of diisocyanate and said third amount (Q₃) of chain extender are present in said mixture in a molar ratio Q₁:Q₂:Q₃ which can range between 1:2:1 and 3:8:5.

14. Composition according to claim 1, wherein a liquid phase in which said polymer is solubilised is distilled water, or a saline solution, or a glucosed solution, or a gluco-saline solution.

15. Composition according to claim 1, wherein the injectable formulation comprises at least one sixth amount (Q₆) of at least one drug or a bioactive molecule.

16. Composition according to claim 15, wherein said sixth amount (Q₆) is included between 0% and 30% weight/volume, more preferably between 0% and 20%, of said solution.

17. Use of a composition according to claim 1 for tissue regeneration and in aesthetic add prosthetic medicine.

18. Use according to claim 17, of said composition as filler, or as vehicle of drugs, growth factors or cells.

19. Polyurethane solution comprising at least one composition according to claim 1, wherein it comprises a fourth amount (Q₄) of polyurethane, included between 1% and 99%, more preferably between 1% and 50%, in weight/volume of said solution.

20. Solution according to claim 19, wherein it comprises a fifth amount (Q₅) of at least one natural polymer and/or carbohydrates and/or proteins.

21. Solution according to claim 20, wherein said natural polymer and/or carbohydrates and/or proteins is chosen among ialuronic acid, gelatine, collagen.

22. Solution according to claim 20, wherein said fifth amount (Q₅) is included between 0% and 99%, more preferably between 1% and 20% in weight/volume of said solution.
